(19) United States
(12) Patent Application Publication
(10) Pub. No.: US 2018/0280266 A1
(43) Pub. Date: Oct. 4, 2018

(54) XANTHOPHYLL COMPOSITIONS FOR SKIN LIGHTENING

(71) Applicant: OmniActive Health Technologies Limited, Mumbai (IN)

(72) Inventors: Jayant DESHPANDE, Charlottetown (CA); Abhijit BHATTACHARYA, Morristown, NJ (US); Vijaya JUTURU, Morristown, NJ (US)

(21) Appl. No.: 15/763,735

(22) PCT Filed: Sep. 28, 2016
(86) PCT No.: PCT/IB2016/055809

§ 371 Date: Mar. 27, 2018

(30) Foreign Application Priority Data
Sep. 28, 2015 (IN) 3663/MUM/2015

(51) Int. Cl. A61K 8/34; A61Q 19/02; A61Q 19/08

(52) U.S. Cl. CPC A61K 8/345; A61Q 19/08; A61Q 19/02

(57) ABSTRACT

Xanthophyll compositions and their methods of use are described, where the compositions include macular pigments as lutein and/or zeaxanthin isomers, and where they are administered in an effective amount for skin lightening and/or improving overall skin tone and/or health in a subject in need thereof. Methods for treatment are described for improvement and/or protection of skin from effects of light rays of variable intensity and/or wavelength on skin tone and/or skin health. Methods herein include administering a xanthophyll composition for skin lightening and/or improving skin tone and/or overall skin health, and to block the melanin pathway and formation of melanin in melanocytes. The compositions and their administration exhibit increased Individual Typological Angle and minimum erythemal dose, which results in rapid skin lightening. The compositions improve overall skin tone and/or skin health, when administered in an effective amount by improving hydration, flexibility and/or protecting skin from damaging effects of light radiation.

### FIELD

Xanthophyll compositions herein include a combination of macular pigments, such as for example lutein and/or zeaxanthin isomers and their methods of use are described for example as administered in an effective amount for skin lightening and improving overall skin tone and health. More particularly, methods of treatment, improvement and protection of skin from effects of light rays of variable wavelengths are described which include administering a xanthophyll composition with an effective amount, such as at least 1 mg of trans-lutein and at least 0.2 mg zeaxanthin isomers in combination. The compositions herein include xanthophylls such as lutein, zeaxanthin, neoxanthin, violaxanthin, flavoxanthin, alpha-cryptoxanthin and betacryptoxanhtin, their isomers, and/or combination thereof. Xanthophyll compositions can be formulated using at least one pharmaceutical, nutraceutical or food grade excipient. The methods described herein include administering a xanthophyll composition to block the melanin pathway and formation of melanin in melanocytes, and are useful for skin lightening and improvement of skin tone. The compositions also exhibit increased Individual Typological Angle and minimum erythemal dose, which can result in rapid skin lightening. The compositions improve overall skin tone, when administered in an effective amount by oral administration. Further xanthophyll compositions described herein also improve skin complexion by improving hydration, flexibility and protect skin from damaging effects of light radiations, which can improve skin tone and cause skin lightening.

### BACKGROUND

Skin acts as a protective membrane and barrier for the body and so healthy skin or epidermis is important for overall well-being. Skin helps to maintain adequate water amount in the body, to avoid drying out or a flooding of internal vital organs. The skin barrier also works to keep out chemicals, pathogens, and sunlight. The skin also determines external look or appearance of a subject, such as the colour, tone, suppleness, complexion, smoothness, moisture level and the like, which one may consider significant to well-being. Skin colour and tone has been a matter of concern for example for women and having good skin colour and tone is desired.

Although skin acts as a barrier, it is exposed to environmental factors like sunlight, dust, smoke, and other irritants. It is delicate and prone to disorders, which can affect skin health. Other factors such as aging, hormonal changes and nutritional imbalance also lead to undesirable changes in skin appearance. When exposed to excessive sun rays, the skin produces more of a pigment called melanin, which darkens the skin and helps protect the body from potentially damaging light rays of varying wavelengths. Such rays penetrate the atmosphere and play an important role in conditions such as premature skin aging, skin pigmentation and/or skin cancers. Constant exposure to light rays of high frequency and intensity damages and/or and can kill skin cells such as in the upper skin layer. In a somewhat mild form, sunburn results in a reddening of the skin called erythema. Prolonged exposure to sun can cause wrinkles, pigmentation, and age spots on the skin. Over time, high intensity light rays damage the fibers in the skin called elastin, causing wrinkles because of loss of tone and elasticity and increased fragility. Further the skin becomes coarse and leathery due to excessive dryness which affects skin tone, hydration and/or complexion.

Localized over-production of melanin due to exposure to sun, pollution, and hormonal changes can lead to hyper-pigmentation. This is often associated with hypo-pigmentation (white spots), which indicates aging due to a reduction in the number of melanocytes. The result is a mottled, older skin appearance with some darker and depigmented areas in between, thus resulting in uneven tone. Skin also experiences dehydration and loss of turgidity, which contributes to altered elasticity in aging skin due to diminishing amounts of hyaluronic acid found in the dermis.

Even though harmful effects from light rays, pollutants, and pathogens can be minimized by avoiding contact with these agents, it is not practical to completely avoid them, because of certain life styles. Therefore many topical products or cosmetic products are used to treat skin disorders or to maintain beautiful skin. These products mainly take care of skin colour, hydration, acne problems or aging, when applied topically to affected areas. Skin depigmentation and lightening products in some cases seem to be a symbolizing target of fashion, seduction, urbanity and/or femininity, rather than to be accepted in society. Cosmetic treatments such as laser treatments are also available by dermatologists to remove the spots more rapidly. Products like sun filters are also used to a great extent, and which play the role of stopping damaging effect of light rays on the skin. However, these products, which need to be applied on the skin quite frequently, also need to be safe, efficient, and produce visible brightening results. Women for example also employ bleaching products on the visible parts of their bodies. Such products not only stop the melanin synthesis process, but they also block the existing melanin to prevent its migration to the surface of the skin. Long term use of such products for improving skin tone, complexion and colour may have side effects such as infections, severe irritation, irreversible skin damages, discoloration, skin cancer, and/or other medical problems. Further currently available skin lightening products may be inefficient and may be harmful to the skin, since a continuous external application of these products can lead to permanent leucoderma and side effects such as dyschromatosis and rash.

Hence there is a need for alternate and safe treatment methods for improving and maintaining desirable skin attributes and for lightening of the skin.

Several natural products are used in cosmetics for topical application if skin is already pigmented (either naturally or after a sun exposure). Cosmetic products containing active molecules such as kojic acid, arbutine, and/or vegetable extracts (such as lemon and/or cucumber rich of Vitamin C) have been used to hinder the process of melanin production and/or stimulate the exfoliation of the skin to eliminate the surface cells containing melanin. Several antioxidant compounds that can inhibit the production of melanin are also utilized in skin whitening products. Since the synthesis of melanin involves an oxidation reaction, blocking the oxidation at various points from tyrosine/dopa to melanin ultimately inhibits the synthesis of melanin. For example, L-ascorbic (Vitamin C) acts as a reducing agent on melanin intermediates and blocks oxidative reactions; other antioxidants utilized by lightening products include bioflavonoids, which are typically extracted from mulberry or licorice.

However as topical products require reapplication and have localized effects, dietary methods are thought to have potential for skin protection and provide continual whole body protection. Thus the potential for dietary skin protection is a relatively recent concept and this has led to introduction of nutrient containing products which are marketed for skin health improvement.

Common ingredients of these products, both cosmetic and consumable, are antioxidant nutrients. A variety of phytonutrients such as α-tocopherol, and certain flavonoids and β-carotene, lycopene, and lutein have been implicated in skin health to combat with oxidative stress and the prevention of skin damage. Carotenoids are known for their antioxidant property and can protect the body against cellular damage, the effects of aging, and even some chronic diseases. As these beneficial compounds cannot be synthesized by humans or animals, it is through the diet that they are obtained. Epidemiological studies have provided some evidence that fruit and vegetable intake may improve skin health and decrease the risk of skin cancer. However, in many cases, the specific effect of an individual carotenoid was not evaluated. Nonetheless, reports have been provided, which support a role for carotenoids, and other phytonutrients, in skin health.

There are references that discuss the use of carotenoids in skin treatment applications.

U.S. patent application US20070098820 provides the use of a primary composition of at least one carotenoid-containing material enriched in cis-isomers, for the preparation of an oral, cosmetic or pharmaceutical composition intended for improving skin health, in particular for photoprotection of the skin or for protecting skin tissue against ageing.

U.S. patent application US20090118228A1 describes a method for improving skin health in a subject includes administering to the subject a combination of lutein, zeaxanthin, lycopene, and beta-carotene, wherein the improvement in skin health comprises a reduction of erythema or rash.

U.S. Pat. No. 8,398,958 describes a method of whitening skin of a subject having undesired skin pigmentation by reducing melanin content within the skin cells of the subject in need thereof, the method includes administering to the subject having undesired skin pigmentation a composition has a combination of phytoene and phytofluene, the combination of phytoene and phytofluene being in an amount sufficient to reduce the melanin content in the skin cells of the subject, thereby whitening the undesired skin pigmentation of the subject by reducing the melanin content within the skin cells of the subject.

U.S. Pat. No. 8,158,164 describes an encapsulated edible emulsion for treating aged skin of 2 to 1000 mg elderberry extracted anthocyanins; and 1 to 20 mg lutein; wherein the weight ratio of elderberry extracted anthocyanins to lutein is at least 1:1.

European patent EP1616556 relates to the use of different mixture of carotenoids, preferably lutein, and gelatin-glycine for improving hydration, elasticity and lipid content of the skin and for protecting skin and ocular tissues from hazardous radiations.

Japanese patent JP4268379B2 describes an oral administration composition, suitable for bleaching the skin containing (a) a carotenoid such as α-carotene, β-carotene, γ-carotene, lycopene, cryptoxanthin, lutein, zeaxanthin, rhodoxanthin, capsanthin, crocetin, or the like and (2) ascorbic acid and/or its salt. However, the patent specifically mentions that oral administration composition is used preferably together with a bleaching cosmetic.

U.S. patent application US20090118228 describes a method for improving skin health in a subject by administering to the subject a combination of lutein, zeaxanthin, lycopene, and beta-carotene; wherein the improvement in skin health is directed to a reduction of erythema or rash.

U.S. Pat. No. 8,715,627 describes an oral and topical formulation of predominantly R-R stereoisomer of zeaxanthin and other carotenoid like lutein to enhance the darkened tint of the skin.

US20090123399 describes a packaged liquid edible composition of a blend of at least two species of fruit and/or vegetables in liquified, puree, pulp, concentrate, extract or juice form, wherein the composition has β-carotene in an amount of 5.5 to 8 mg, lycopene in an amount of 2 to 4 mg, lutein in an amount of 1 to 3 mg.

The composition is for treating the human skin, including skin care, skin condition, skin appearance, skin colour, for a skin bronzing, skin browning, skin colouring effect, UV-protection, UV-B-protection, sun-screen, protection against aging, providing anti-oxidants to the skin, and protection against photo-induced aging.

JP09025209A describes a skin preparation for external use to suppress generation of melanin and effective for preventing and improving of chromatosis after sunburn, spot, freckle, chloasma. The preparation has lutein which is obtained by extracting from a plant such as plant such as Cola-de-Caballo (family Equisetales)

Kemin has published a technical literature which describes important skin studies involving lutein (Richard Roberts, Kemin Foods, KHTL-017-80316 Rev 9), in which FloraGLO oral and/or topical administration resulted in improvement in skin hydration, elasticity, lipid content and photoprotective activity(1998-2007). This literature mentions various research papers related to skin health which are enumerated with some details below:

Palombo paper confirmed effect of lutein and zeaxanthin on reducing detrimental effects of sunlight exposure upon human skin.

Astner demonstrated the potential for lutein and zeaxanthin to protect against detrimental cutaneous photoaging effects of UVB exposure of human skin.

Philips et al. demonstrated that xanthophylls may have beneficial protective effects on the extracellular matrix from degeneration associated with both normal aging of skin and premature aging of the skin caused by UV light exposure.

Lee et al. inferred that dietary lutein reduces UV-induced inflammation and Morganti proved that skin hydration is improved in volunteers having supplements such as lutein, lipoic acid, ascorbic acid and tocopherol.

Gonzalez et al, concluded that oral supplementation of lutein and zeaxanthin diminishes effects of ultraviolet B irradiation by reducing acute inflammatory responses.

Morganti et al. also reported that consumption of an antioxidant product containing 6 mg daily dose of FloraGLO lutein may decrease amount of skin lipid peroxidation while simultaneously increasing both skin lipids and skin hydration.

Stahl. showed that erythema resulting from UV irradiation was significantly reduced after eight weeks of supplementation with a carotenoid mixture containing lutein.

### SUMMARY

Although the above references discuss phytonutrient compositions for treatment of skin ailments such as erythema, skin rash, aging or skin pigmentation, there are no references which describe use of natural product or xanthophylls supplement for skin lightening effect and improvement of skin complexion, tone and flexibility affected due to light rays of variable intensity and wavelengths.

The inventors have carried out exhaustive trials for the evaluation of xanthophyll compositions, which exhibits skin lightening effect and improvement of overall skin health and tone, when administered in an effective amount, such as for example by oral administration. The compositions can also improve skin hydration and/or flexibility, and can protect the skin from environmental effects such as temperature and also from light of variable wavelengths and/or intensity. These compositions herein contain xanthophylls such as lutein and/or zeaxanthin isomers either alone, and in some embodiments in combination with other xanthophylls or their isomers, and where the xanthophylls are derived from plant extracts and/or oleoresin. The methods of administering xanthophyll compositions and the compositions, are safe for human consumption and can be prepared using conventional industrial equipment using economic processes.

In an embodiment, xanthophyll compositions herein, when administered to a subject in an effective amount, are useful for skin lightening and for improving overall skin tone and skin health.

In an embodiment, a method includes administering a xanthophyll composition with macular pigments, including for example lutein and/or zeaxanthin isomers, and evaluating their use for treatment, improvement, and/or protection of skin from effects of light rays of variable wavelengths.

In an embodiment, xanthophyll compositions herein include lutein, zeaxanthin, neoxanthin, violaxanthin, flavoxanthin, alpha-cryptoxanthin, and/or betacryptoxanhtin, their isomers, or combinations thereof, and are useful, for example when administered to a subject, for skin lightening and protection from effects of light rays of variable wavelengths.

In an embodiment, xanthophyll compositions herein include macular pigments, such as for example lutein and/or zeaxanthin, and/or their isomers, which are derived from plant extracts and/or oleoresin containing xanthophylls and/or xanthophyll esters, and which are safe for human consumption.

In an embodiment, a method includes administering a xanthophyll composition including trans-lutein isomer either alone or in combination with one or more zeaxanthin isomers, such as for example (R,R) zeaxanthin and/or (R,S) zeaxanthin, and which is formulated using at least one pharmaceutical, nutraceutical or food grade excipient.

In an embodiment, xanthophyll compositions herein, which are derived from a plant extract and/or oleoresin, are prepared using conventional industrial equipment and economically convenient processes.

In an embodiment, a method includes administering a xanthophyll composition to block the melanin pathway and formation of melanin in melanocytes, and which is useful for skin lightening and improvement of skin tone.

In an embodiment, a method includes administering a xanthophyll composition for example by oral route to exhibit increased Individual Typological Angle and minimum erythemal dose, and which are useful for skin lightening and skin tone improvement effect.

In an embodiment, use of the compositions herein are to improve overall skin tone and skin health, when administered in an effective amount, such as for example by oral administration.

In an embodiment, a method includes administering a xanthophyll composition to improve skin complexion by improving hydration, flexibility and protect skin from damaging effects of light radiations.

In an embodiment, xanthophyll compositions herein are useful for improving skin tone and are safe for human consumption, when administered in an effective amount such as for example by oral administration.

In an embodiment, methods herein can include administering a xanthophyll composition alone such as for example by oral supplementation, or in combination with a topical application, in an effective amount, for skin lightening effect and for improving overall skin tone and elasticity.

In an embodiment, methods herein can include administering a xanthophyll composition such as for example by oral supplementation in an effective amount to improve skin attributes, such as for example for skin hydration and/or complexion, and/or for reducing roughness experienced due to exposure to varying temperature and/or light rays of high intensity and/or variable wavelengths.

In an embodiment, a method for skin lightening of a subject includes administering a xanthophyll composition such as for example by oral administration, in an effective amount, such as for example at least 1 mg of trans-lutein alone or at least 0.2 mg zeaxanthin isomers, such as for example (R,S) zeaxanthin and/or (R,R) zeaxanthin, or in the form of a combination of at least 1 mg trans-lutein and at least 0.2 mg zeaxanthin isomers.

In an embodiment, xanthophyll compositions comprising a combination of macular pigments such as for example trans-lutein and zeaxanthin isomers can be used for skin lightening and for improving skin tone and overall skin health, when administered for example by oral route, in an effective amount to a subject in need thereof. Such subject in need may or may not be exposed to light rays of variable intensity and wavelength, but is suffering from hyper pigmentation or hypo pigmentation. The subject also may be exposed to varying environment conditions of temperature and experiencing rough and uneven skin. Xanthophyll compositions herein include macular pigments such as lutein and/or zeaxanthin, and/or their isomers, either alone or in combination thereof. More particularly, methods described herein relate to administration of xanthophyll compositions including trans-lutein, zeaxanthin isomers, namely, (R,R) zeaxanthin and (R,S) zeaxanthin, either alone or in combination thereof, in an effective amount to a subject in need thereof.

In an embodiment, a method for skin lightening of a subject includes administering a xanthophyll composition, such as for example by oral administration, in an effective amount, such as at least 1 mg of trans-lutein alone or at least 0.2 mg zeaxanthin isomers such as for example (R,S) zeaxanthin and (R,R) zeaxanthin, or in the form of a combination of at least 1 mg trans-lutein and at least 0.2 mg zeaxanthin isomers, for example (R,S) zeaxanthin and (R,R) zeaxanthin.

In an embodiment, xanthophyll compositions herein are used, such as by administration, to block the melanin pathway as well as formation of melanin in melanocytes, which can result in skin lightening and improvement of skin tone. Administration of the compositions herein also exhibits increased Individual Typological Angle (ITA) and minimum erythemal dose (MED).

In an embodiment, methods herein include administering a xanthophyll composition which prevents transepidermal water loss and reduces signs of aging of skin, by the administration of an effective amount to a subject such as for example by oral administration. The compositions described herein also improve overall skin tone, when administered in an effective amount by oral administration. Methods described herein can also result in improved skin hydration, complexion, elasticity, and/or flexibility, and can protect skin from damaging effects of light radiation, to improve overall skin tone, reduce wrinkles, and/or provide luster to skin.

In an embodiment, xanthophyll compositions herein are prepared by economical and industrially viable processes and are useful as a supplement for skin lightening effect.

### BRIEF DESCRIPTION OF THE DRAWINGS

FIG. 1 is a graph that shows the effect on minimum erythemal dose (MED) after lutein and zeaxanthin isomers (L/Zi) supplementation.

FIG. 2 is a graph that shows the effect of xanthophyll composition on overall skin tone.

FIG. 3 is a graph that shows enhancement of elastic recovery based on L/Zi supplementation.

FIG. 4 is a graph that shows the effect of xanthophyll composition administration on transepidermal water loss.

FIG. 5 is a graph that shows the effect of xanthophyll composition administration on skin hydration.

### DETAILED DESCRIPTION

In an embodiment, xanthophyll compositions and methods for their administration to a subject in an effective amount are described which are useful for skin lightening and/or improving overall skin tone and/or health.

Methods described herein are directed to methods of administering xanthophyll composition in an effective amount for prevention, treatment and/or improvement of skin health to a subject in need of such prevention, treatment and/or improvement related to skin health.

The terminology ‘skin health’ refers to maintaining overall skin attributes such as skin colour, tone, hydration, flexibility, even texture, absence of blemishes, rashes, pimples or hypo and/or hyperpigmentation, and/or any other similar type of attributes which indicate healthy and beautiful skin. Skin lightening or improving skin tone is considered to be one of the parameters which are considered by some an important perspective of beauty, for example in Asian countries; however skin lightening or improved skin tone along with other parameters such as soft, supple, even and/or pimple free skin can be considered as indication of healthy and beautiful skin.

The term ‘skin lightening’ as used herein refer to improvement of skin tone or colour from existing one to fair or lighter shade. Although this is relative term, it is considered that skin lightening is the method in which dark shade skin is gradually converted to lighter or fair one. Skin lightening effect by some is desired, such as for example in beauty conscious individuals.

The terminology ‘subject’ is commonly used in the specification to refer to an individual or human being under test, being administered with xanthophyll compositions described herein.

The terminology ‘subject in need thereof’ can include specific individuals or human beings, who are getting exposed to various factors such as pollutants, temperature fluctuations, dry weather, light rays of variable intensity and/or wavelength, hormonal changes, and/or chemicals and the like or the combination thereof. These can affect various skin attributes such as for example skin tone, colour, complexion, and/or texture and can result in hyper-pigmentation and/or faster skin aging and damage to skin, which can also affect skin colour as well as skin health.

Xanthophyll compositions and methods described herein can lighten the skin and also improve skin attributes such as skin tone, hydration, roughness and/or overall complexion, and/or can protect skin from effects of light sources of variable intensity and/or wavelengths, when administered to a subject, in need of such treatment.

The term ‘light sources of variable intensity and wavelengths’ as used herein refers to light rays emerging from various sources such as the sun, electronic instruments, fire, domestic light sources, and/or rays emerging from household cooking energy sources, or any other sources which give rise to light rays of low, medium, or high intensity and varying wavelengths, such as near, medium, long causing blue light, ultraviolet light or white light spectrum of light. Such light sources give out light rays which become incident on skin surface and may get absorbed and therefore causing visible, invisible, short term or long term damage to the skin. These may result into darkening of the skin, creating blemishes, seen as hyper- or hypo-pigmentation or may cause rashes, thus affecting overall appearance and health of skin, resulting into ageing and dull skin.

Skin colour and/or overall skin tone may get affected due to various internal and/or external factors, such as for example lack of proper nutrition and water, lack of sufficient sleep, stress factors, wrong eating habits such as oily and junk foods, exposure to light rays of varying intensity, pollutants, dry weather and/or hormonal changes. It is important to treat affected skin at early stage. Moreover having lighter skin and tone may be desired in beauty conscious individuals, such as for example in certain cultures including Asian and African cultures, and including individuals in countries such as for example Japan, China, India and in the Middle East region, where many topical creams and face powders have been used on a routine basis. However delicate skin gets exposed to chemicals used in beauty creams or powders, and may result in blemishes, pimples, roughness and/or allergic reactions. Therefore it may be preferable to use naturally-sourced beauty products or opt for systemic treatment using natural supplements instead of using topical cosmetic products. A balanced diet and plenty of fluids is also an option for skin lightening and maintaining overall skin tone and health. Lutein is one of the carotenoids which is found in diet that is present in high concentrations in skin cells and also in the eye and is also known as macular carotenoid. Lutein and zeaxanthin isomers are powerful antioxidants and possess property of absorbing light in ultraviolet range.

The compositions described herein are safe for use and are meant for administration either through oral route or topical use in combination with oral route. More particularly, xanthophyll compositions herein include macular pigments such as for example lutein and/or zeaxanthin isomers, either alone or specifically in combination thereof, and wherein their uses are for methods of administration for skin lightening and/or for improving overall skin tone and health.

In an embodiment, the methods described herein use xanthophyll compositions comprised of macular pigments such as for example trans-lutein and/or zeaxanthin isomers, including (R,R) zeaxanthin and (R,S) zeaxanthin, either alone or in combination thereof, in a specific ratio, for the purpose of skin lightening and improving overall skin health, when administered to a subject in an effective amount. The compositions described as per the methods herein, are also employed for prevention, treatment and improvement of skin health, when exposed to light sources of variable intensity and wavelength.

In an embodiment, xanthophyll compositions described herein are derived from plant extracts and/or oleoresins and are safe for human consumption. The compositions herein are prepared using conventional equipment and economic processes.

In an embodiment, methods as described herein make use of xanthophyll compositions comprised of lutein including trans-lutein and/or zeaxanthin isomers, either alone or in combination thereof, and these are administered in an effective amount to a subject in need of skin lightening or improvement of skin tone and overall skin health.

In an embodiment, methods for skin lightening or improving overall skin health make use of compositions comprised of lutein and/or zeaxanthin isomers alone or in combination thereof. The isomers of lutein used in such compositions is preferably trans-isomer and zeaxanthin isomers can be (R,R) zeaxanthin or (R,S) zeaxanthin, used either alone or in combination thereof.

In an embodiment, the compositions described herein are comprised of trans-lutein and zeaxanthin isomers in the range of about 4:1 to about 6:1 and the ratio of the isomers of zeaxanthin [(R,R) zeaxanthin and (R,S) zeaxanthin] being in the range of 80 to 20:20 to 80.

In an embodiment, the process for preparation of the xanthophyll composition includes mixing oleoresin or extract containing xanthophyll esters with 1-propanol. The mixture is heated to a temperature of 90 degree C. and maintained for 1-5 hours, under agitation. After obtaining the desired degree of isomerization and the xanthophylls composition with trans-lutein content at least 85%, the reaction mixture is diluted with water and stirred well at room temperature. After transferring this oily layer in a separatory funnel, ethyl acetate is added and the xanthophylls are extracted. The ethyl acetate layer is washed twice with an equal volume of de-ionized water. The ethyl acetate extract is concentrated by di stilling off the solvent under reduced pressure to recover ethyl acetate and the crude xanthophyll concentrate. The xanthophyll composition is subjected to purification by agitating with hexane at room temperature for one hour, followed by filtration. The xanthophyll mass is further washed with ethanol and the resulting orange crystals are dried under vacuum at ambient temperature for 72 hours.

In an embodiment, xanthophyll compositions described herein are formulated using at least one pharmaceutical, nutraceutical or food grade excipient. The excipient can be a carrier, granulating agent, inert core, coating agent, solvent, oil medium, diluents, carrier, binder, lubricant, disintegrant, antioxidant, oil, surfactant, solubilizer, emulisifer or any other excipient, which is known to a person skilled in the art as excipient required for preparing palatable dosage form, acceptable to the subject.

In an embodiment, methods described herein use xanthophyll compositions for skin lightening and improving skin tone, wherein the compositions by their administration block the melanin pathway and formation of melanin in melanocytes. The compositions, when administered, also exhibit increased Individual Typological Angle and minimum erythemal dose, thus resulting into rapid skin lightening.

In an embodiment, the compositions herein also improve overall skin tone, when administered in an effective amount by oral administration. Further, xanthophyll compositions described herein, when administered, can also improve skin complexion by improving hydration and flexibility, and also help to protect skin from damaging effects of light radiations, which can improve skin tone and cause skin lightening.

In an embodiment, xanthophyll compositions herein may be administered to a subjects in need thereof, in an effective amount, such as for example by oral administration, for skin lightening. The oral administration may be also combined with topical application of xanthophyll compositions for skin lightening and improving skin tone.

In an embodiment, methods herein include administering a xanthophyll composition including macular pigments, such as for example trans-lutein and/or zeaxanthin isomers, in an effective amount to a subject in need thereof.

In an embodiment, the composition is administered in an effective amount so that at least 1 mg of trans-lutein is administered either alone or in combination with at least 0.2 mg of zeaxanthin isomers, such as for example (R,R) zeaxanthin and (R,S) zeaxanthin. When trans-lutein is administered in combination with zeaxanthin isomers, ratio of (R,S) zeaxanthin:(R,R) zeaxanthin can be 80:20 to 20:80.

In an embodiment, compositions herein are administered in an effective amount so that about 5 mg of trans-lutein is administered either alone or in combination with about 1 mg of zeaxanthin isomers, such as for example (R,R) zeaxanthin and (R,S) zeaxanthin.

In an embodiment, compositions herein are administered in an effective amount so that about 10 mg of trans-lutein is administered either alone or in combination with about 2 mg of zeaxanthin isomers, such as (R,R) zeaxanthin and (R,S) zeaxanthin.

In an embodiment, xanthophylls composition herein are administered in an effective amount so that about 40 mg of trans-lutein is administered either alone or in combination with about 8 mg of zeaxanthin isomers, such as for example (R,R) zeaxanthin and (R,S) zeaxanthin.

In an embodiment, xanthophylls composition herein may be administered in an effective amount so that about 1 to 40 mg of trans-lutein or 0.2 to 8 mg of zeaxanthin isomers is administered to a subject for use as skin lightening or for improving skin tone and/or overall skin health.

In an embodiment, a ratio of zeaxanthin isomers, including meso-zeaxanthin and R,R zeaxanthin, is about 75:25, when administered in combination with trans-lutein to the subject in need thereof.

In an embodiment, the effective amount may be given as an oral supplementation or as a combination of oral and topical administration to a subject in need thereof.

In an embodiment, xanthophyll compositions herein may be administered to a subject, along with other nutrients such as fatty acids and at least one food grade excipient such as, but not limited to, diluents, carrier, binder, lubricant, disintegrant, antioxidant, oil, surfactant, solubilizer, emulisifer, and the like or the combinations thereof.

In an embodiment, xanthophyll compositions herein may be administered to a subject, in the form of suitable solid or liquid dosage form in an effective amount, for skin lightening and for improvement of overall skin tone and skin health.

In an embodiment, a method includes administering an effective amount of a xanthophylls composition for skin lightening, wherein the composition prevents melanin production.

In an embodiment, xanthophyll compositions herein and methods herein for administering a xanthophyll composition in an effective amount are useful for skin lightening through increase in minimum erythemal dose (MED) and Individual Typological Angle (ITA).

In an embodiment, xanthophyll compositions herein were evaluated in human volunteers under different experimental conditions, to check skin lightening effects of the xanthophylls compositions herein and to check their effect on minimum erythemal dose.

### Example 1: To Evaluate the Efficacy of Xanthophyll Composition for Skin Lightening and Skin Health

The objective of the study was to investigate the efficacy of xanthophylls composition relative to a placebo in improving skin colour, photo-protective activity, and overall skin tone in healthy male and female subjects.

Forty six subjects including male and female with mild to moderate dry skin, Fitzpatrick Skin Type II-IV scale (medium to brown skin colour) and normal healthy individuals were recruited into a double-blinded, randomized, placebo controlled trial over 12 weeks intervention period.

Baseline Characteristics of the subjects were recorded before starting the experiment. Subjects were orally administered with xanthophyll composition (combination of 10 mg lutein+2 mg of zeaxanthin isomers such as R,R zeaxanthin and meso-zeaxanthin) in daily dose in the form of oil suspension using safflower oil as vehicle. Remaining subjects were orally administered with a placebo (safflower oil) one time per day. The group administered with xanthophylls composition is called as actives group and the other group is called as placebo group, as mentioned in examples part. The right forearm was exposed to 60 mJ/cm² UVA/UVB light [280-320 nm) from a 150 watt solar stimulator. Various imaging techniques can be used for assessing redness, tanning and overall skin colour. The skin surface was assessed with a Minolta Chromameter CR-400, after 24 h of this exposure to check colour parameters such as L, A and b. The photo protective activity values were measured in terms of MED and skin colour. The minimal erythemal UV dose (MED) for treated skin was determined during the study. The Canfield VISTA-CR® 2.2 Facial Imaging Booth w/Canon 5D Professional Digital body was used to provide photographs of the face during the study. The hardware was controlled and images were data-based using Canfield software. Other parameters like trans-epidermal water loss (TEWL), hydration, skin elasticity, skin roughness and skin aging attributes were also evaluated to check effect of xanthophylls composition after 12 weeks administration.

All volunteers were checked for baseline characteristics and the details are recorded in Table 1.

TABLE 1
Baseline characteristics of the Volunteers

| Details | Placebo | Actives |
|---|---|---|
| Age, y | 37.6 ± 6.0 | 36.0 ± 7.8 |
| Gender, M/F | 3 Males + 18 Females | 4 Males + 23Females |
| Body mass | 26.05 ± 3.57 | 26.83 ± 3.6 |
| index(BMI), Kg/m2 | | |
| Systolic blood pressure | 114 ± 11.13 | 111 ± 12.19 |
| (BP), mm Hg | | |
| Diastolic BP, mm Hg | 73 ± 7.93 | 72 ± 10.43 |
| Heart rate (HR), Beats | 70 ± 10.92 | 67 ± 10.55 |
| per minutes | | |
| Respiratory rate, | 16 ± 1.99 | 18 ± 1.87 |
| Breaths per minute | | |
| Oral temperature, ° F. | 98.51 ± 0.43 | 98.45 ± 0.55 |

A. Evaluation of Effect of Xanthophyll Composition Administration on Skin Colour

Various imaging techniques can be used for assessing redness, tanning and overall skin colour. For this purpose colour space system L*a*b* is used for measurement. It measures the skin colour as L* Lightness, a* redness and b* yellowness. An increase in L* and a decrease in b* parameters is indicative of skin lightening. Individual typology angle (ITA) was also estimated as a tool for predicting related clinical effects of ultraviolet radiation (UVR) exposure. The ITA value allows an objective skin colour type classification that is potentially predictive of the biological consequences of UVR exposure. ITA and skin colour was evaluated in actives group and placebo group and the outcomes are recorded in Table 2.

TABLE 2
Evaluation of skin colour using chromameter

| | Placebo | | | L/Zi (Actives) | | |
|---|---|---|---|---|---|---|
| | | | Difference | | | Difference |
| | | Week | from | | | from |
| Area | Baseline | 12 | baseline | Baseline | Week 12 | baseline |
| L* | 62.02 | 62.59 | 0.57 | 62.04 | 63.08 | 1.23* |
| | | | (0.3216) | | | (0.0094) |
| b | 16.50 | 15.94 | −0.56* | 16.22 | 15.35 | −1.05* |
| | | | (0.03) | | | (0.0013) |
| ITA° | 35.91 | 37.79 | 1.88 | 36.3 | 39.97 | 3.67* |
| | | | (0.0754) | | | (0.0009) |

*significance over baseline; p value in parenthesis; L: Lutein and Zi: Zeaxanthin isomers

After 12 weeks of xanthophyll composition administration, overall skin ITA increased in actives over placebo (P<0.0237) and the luminance (L*) values were significantly increased from baseline (63.08, p<0.0094) compared to placebo (62.59, p<0.0.3216). Also b* values significantly decreased (−0.56 vs −1.05). Table 2 indicates that all values in volunteers administered with xanthophylls composition for 12 weeks were statistically significant for all three parameters L, b and ITA. The composition exhibited decrease in b values and increased ITA and L values, thus it was able to provide rapid skin lightening. The composition also exhibited good compatibility and was well tolerated in all volunteers.

B. Effect of Xanthophyll Administration on Minimal Erythemal Dose (MED)

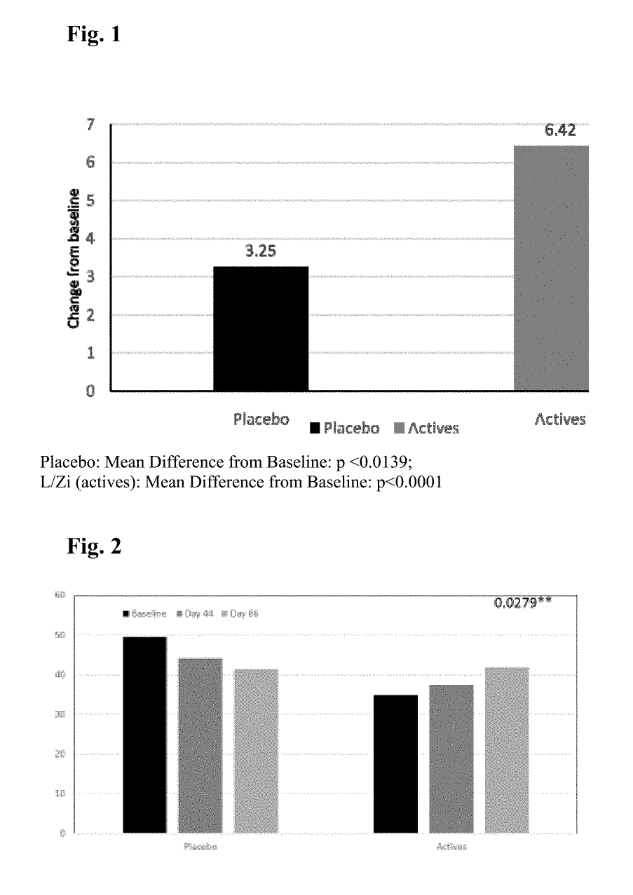

The dosage of UV light is prescribed according to an individual's skin sensitivity. Thus, to establish the proper dosage of UV light to administer, the volunteer has to be screened to determine a minimal erythemal dose (MED), which is the amount of UV radiation that will produce minimal erythema (sunburn or redness caused by engorgement of capillaries) of an individual's skin within a few hours following exposure. In this study MED was determined in all volunteers and then effect of xanthophylls composition or placebo is evaluated over a period of 12 weeks. It was observed that mean MED was significantly increased in actives group as compared to placebo group (3.25 vs 6.42) as shown in FIG. 1. This indicates that the composition increased resistance of the skin to exposure of light rays of varying intensity and wavelength, when administered in effective amount as a daily dose.

C. Subjective Evaluation of Effect of Xanthophylls Composition on Skin Tone

The groups administered with active (xanthophylls composition) and placebo were observed for changes in skin tone over a period of 12 weeks. During subjective assessment, it was observed that there was a significant difference on Overall Skin Tone at Day 86. FIG. 2 indicates that overall skin tone in subjects administered with xanthophyll composition (3.15/7.17, p<0.05) was significantly improved as compared to subjects administered with placebo (−5.45/−8.20, p<0.1), P<0.0237 between treatments).

D. Effect of Xanthophylls Composition Administration on Skin Elasticity

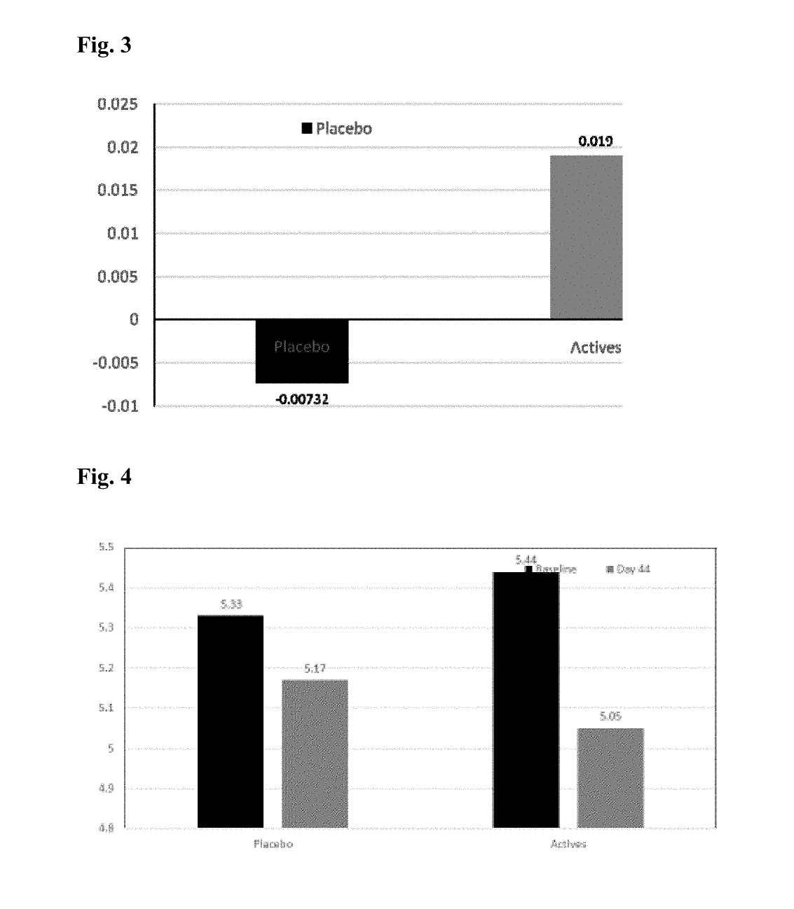

Cutometer was used to determine effect of xanthophyll composition on skin elasticity and its comparison with effect of placebo on skin elasticity. This instrument is designed to measure elasticity of upper skin layer using negative pressure which deforms the skin mechanically. Elastic recovery effect was checked in volunteers administered with active (xanthophylls composition) and also in volunteer group administered with placebo. As shown in FIG. 3, it was observed that elastic recovery in actives was (0.02, p<0.0132), while in group administered with placebo it was (−0.01, p<0.0159). This indicates that volunteers administered with actives exhibit greater increase in extensibility and faster elastic recovery of the skin, which also confirms that firmness of skin is enhanced in actives treatment.

E. Effect of Administration of Xanthophyll Composition on TEWL and Skin Hydration

TEWL (Trans-epidermal Water Loss) measurements are used to characterize the skin water barrier function and are considered an appropriate measure to be used in human intervention studies assessing an intervention's ability to protect the skin against dehydration (Pinnagoda et al., 1990; EFSA 2012). TEWL is the amount of water that diffuses through the skin and evaporates from the skin surface and is measured in g/m²/h. TEWL can be measured by a measurement device (DermaLab, TEWL Cortext technology) that measures the moisture in skin.

TEWL was measured on the legs (Pinnagoda et al., 1990). Subjects were asked to be in a rested state with their surface of examination (i.e., ventral surface of the leg) exposed. The TEWL was measured on the left leg. The same leg was measured throughout the study.

TEWL was measured in the group administered with actives (lutein and zeaxanthin isomers) and another group administered with placebo. As shown in FIG. 4, it was observed that trans-epidermal water loss decreased at week 6 in subjects administered with actives (−0.51 vs −0.17, p<0.0528). It was also observed that there were 74.1% responders in active group and 65% responders in placebo at week 6 to TEWL measurement. Further it was seen that there were 35% responders in placebo and 37.5% responders in actives at week 12.

A Den Lab Moisture Meter (Cortext Technology) with a high sensitivity flat probe was employed to measure skin hydration. The recorded value represents the AC conductance 2 seconds after placing the spring-loaded probe tip to the middle of the application site.

Corneometry measurements included 3 conductance readings taken from each site. These 3 readings were taken at each application site and then averaged. An averaged value was computed at each time point. Corneometer measurements were taken immediately following TEWL measurements. Measurements were made on the leg adjacent to the site used for TEWL measurements.

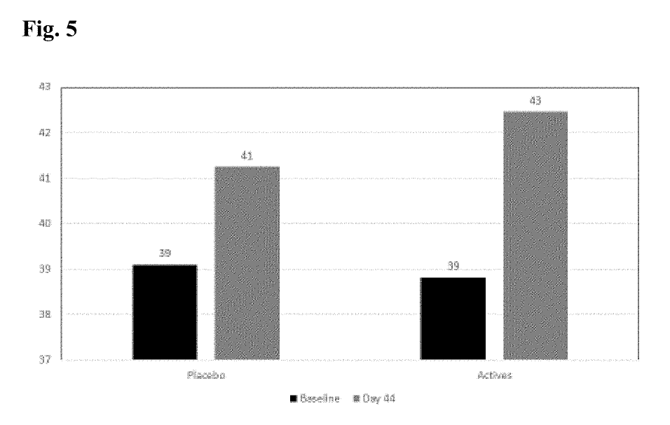

Right leg of volunteers was used for this assessment. It was observed that as per FIG. 5, hydration increased in actives at week 6 (4.48 vs 2.15, p>0.5). There were 66.7% responders in actives and 40% responders to placebo (p<0.0837). Only 33.3% responders were observed in actives and 40% responders to placebo group (p>0.5).

F. Subjective Assessment of Effect of Xanthophyll Composition on Skin Attributes (Skin Aging and Skin Tone Clarity)

Skin Aging:

The results of the observation indicated significant changes from baseline in active treated group for facial fine lines at Day 44 and Day 86 and for pore appearance of the Face at Day 86. The analysis also indicated that there was a significant difference in radiance and skin aging observed at day 86 in active and placebo treated group.
Radiance: Actives (2.17) vs placebo (0.20) p>0.5
Skin aging: Actives (1.00) vs placebo (0.23), p>0.5

Skin Tone Clarity:

The results of the analysis indicated significant changes from baseline for Skin Tone Clarity at Day 86, for Skin Tone Evenness at Day 86 and for Wrinkles for Treatments at Days 44 and 86.
Skin firmness: Actives (3.96) vs placebo (−30), p<0.2431
Skin tone evenness: Active (6.96, p<0.0106) vs placebo (3.85, p<0.116) at week 12
Wrinkles Actives (6.56/4.29, p<0.05) vs placebo (5.25/6.95, p<0.05)

The results of subjective assessment are recorded in Table 3.

TABLE 3
Subjective assessments for skin attributes

| Area | Placebo | L/Zi (actives) |
|---|---|---|
| Facial Discolouration | −1.20 | 9.87* |
| Facial Lines and Wrinkles | 7.75 | 4.87 |
| Overall Skin Tone** | −8.20 | 7.04 |

*Significant change from baseline
**Significant changes between the treatments L/Zi (actives)
It was observed that overall skin tone was improved in group treated with xanthophyll composition (lutein and zeaxanthin isomers-R,R zeaxanthin and meso-zeaxanthin combination) compared to placebo treatment (−8.20 vs 7.04, p < 0.0237).

Thus from above results it is seen that skin hydration was significantly improved after supplementation in actives at week 6. TEWL was decreased in actives in responders. There were no significant changes in both the parameters at week 12. Skin tone evenness was significantly increased in active group (6.96, p<0.0106) vs placebo (3.85, p<0.116) at week 12. Wrinkles decreased over baseline (week 6/week 6 from baseline score) in actives (6.56/4.29, p<0.05) vs placebo increased over baseline (5.25/6.95, p<0.05). Greater increase in extensibility and faster elastic recovery of the skin indicated more firmness of the skin in active treatment. Oral supplementation of xanthophyll composition comprising combination of trans-lutein, zeaxanthin and meso-zeaxanthin resulted into improved overall skin tone and colour. Positive effect was also observed in active treated group in improved overall skin tone, lightness ITA° and increase in MED, indicating beneficial effects of xanthophyll compositions on skin health.

We claim:

1. A method for treating skin, comprising administering a xanthophyll composition including a combination of macular pigments which, when administered in an effective amount to a subject, the administering results in skin lightening and improvement of skin tone and health.

2. The method of claim 1, wherein the macular pigments include lutein and zeaxanthin isomers, and the administering improves skin tone by treating skin from light rays of varying intensity and wavelengths.

3. The method of claim 1, wherein the administering lightens the skin by blocking melanin pathway and formation of melanin in melanocytes.

4. The method of claim 2, wherein the lutein includes trans-lutein and the zeaxanthin isomers include R,R-zeaxanthin and R,S-zeaxanthin, which lightens the skin through increase in Individual Typological Angle and increase in minimum erythemal dose.

5. The method of claim 1, wherein the administering improves overall skin tone by enhancing luminescence value and reducing yellowness value.

6. The method of claim 1, wherein the administering improves skin health by enhancing skin elasticity and skin hydration.

7. The method of claim 1, wherein the administering which improves skin health by reducing skin aging attributes such as facial lines, wrinkles, and face discoloration and by enhancing overall skin tone.

8. The method of claim 4, wherein the effective amount includes at least 1 mg of trans-lutein combined with at least 0.2 mg of zeaxanthin isomers as a daily dose, where the zeaxanthin isomers including R,R zeaxanthin and R,S meso-zeaxanthin.

9. The method of claim 4, wherein the effective amount includes about 10 mg of trans-lutein combined with about 2 mg of zeaxanthin isomers as a daily dose, where the zeaxanthin isomers including R,R zeaxanthin and R,S meso-zeaxanthin.

10. The method of claim 4, wherein the effective amount includes about 40 mg of trans-lutein combined with about 8 mg of zeaxanthin isomers as a daily dose, where the zeaxanthin isomers including R,R zeaxanthin and R,S meso-zeaxanthin.
